(19) United States
(12) Patent Application Publication
(10) Pub. No.: US 2024/0387022 A1
(43) Pub. Date: Nov. 21, 2024

(54) MEDICAL INFORMATION PROCESSING APPARATUS AND MEDICAL INFORMATION PROCESSING SYSTEM

(71) Applicant: CANON MEDICAL SYSTEMS CORPORATION, Otawara-shi (JP)

(72) Inventors: Takashi HYO, Nasushiobara (JP); Yoshihiro NODA, Utsunomiya (JP); Kenichi USUI, Nasushiobara (JP); Takao FUKIAGE, Otawara (JP); Daigo OKUHATA, Sakura (JP)

(73) Assignee: CANON MEDICAL SYSTEMS CORPORATION, Otawara-shi (JP)

(21) Appl. No.: 18/663,295

(22) Filed: May 14, 2024

(30) Foreign Application Priority Data
May 15, 2023 (JP) 2023-080377

(51) Int. Cl. G16H 40/00; G16H 10/60; G16H 15/00

(52) U.S. Cl. CPC G16H 40/00; G16H 10/60; G16H 15/00

(57) ABSTRACT

In one embodiment, a medical information processing apparatus includes processing circuitry. The processing circuitry acquires a plurality of medical support information items regarding a patient and a generation-time information item from a patient device to be operated by the patient, the generation-time information item indicating respective clock times at which the medical support information items are generated, generates timeline data indicating a temporal flow of generation of the medical support information items regarding the patient by arranging the medical support information items in accordance with the generation-time information item, and displays the timeline data on an online-medical-care support screen for supporting online medical care that is performed via the patient device and a doctor device to be operated by a doctor.

### CROSS-REFERENCE TO RELATED APPLICATION

This application is based upon and claims the benefit of priority from Japanese Patent Application No. 2023-080377, filed on May 15, 2023, the entire contents of which are incorporated herein by reference.

### FIELD

Disclosed embodiments relate to a medical information processing apparatus and a medical information processing system.

### BACKGROUND

Recently, online medical care has become popular. The online medical care has merits in many cases such as medical care during the COVID-19 pandemic, medical care during childcare, initial diagnosis in a remote place, providing a second opinion for a patient in a remote place, and sudden night-time illness of an infant.

However, the online medical care lacks information that doctors can use. Thus, there are many cases where the patient is required to come to the hospital for a thorough examination and cases where the online medical care cannot adequately treat the patient in the first place.

For a patient, the online medical care is different from face-to-face medical care because the patient can only see a doctor on a relatively small screen such as a smartphone. Hence, it is difficult for a patient to receive convincing explanation from a doctor. In reality, the online medical care is often used only for follow-up monitoring of a patient whose symptoms are stable, and the online medical care is difficult to be applied for other purposes.

Doctors can acquire information equivalent to the past medical records at other medical institutions by linking the online medical care system with a regional medical cooperation system. However, the regional medical cooperation system does not usually record detailed conditions such as the patient's detailed progress and daily behavior, and thus, the doctors cannot acquire information on a detailed condition of the patient.

### BRIEF DESCRIPTION OF THE DRAWINGS

In the accompanying drawings:

FIG. 1 is a block diagram illustrating a configuration of a medical information processing system according to the first embodiment;

FIG. 2 is a schematic diagram illustrating an outline of processing to be performed by the medical information processing system according to the first embodiment;

FIG. 3 is a sequence chart illustrating processing to be performed by the medical information processing system according to the first embodiment;

FIG. 4 is a schematic diagram illustrating an online-medical-care support screen according to the first embodiment;

FIG. 5 is a schematic diagram illustrating a case of showing a reliability degree of each patient information item on the online-medical-care support screen according to the first embodiment;

FIG. 6 is a schematic diagram illustrating another case of showing the reliability degree of each patient information item on the online-medical-care support screen according to the first embodiment;

FIG. 7 is a schematic diagram illustrating a case of showing a contribution degree of each patient information item on the online-medical-care support screen according to the first embodiment; and

FIG. 8 is a block diagram illustrating a configuration of a medical information processing system according to the second embodiment.

### DETAILED DESCRIPTION

Hereinbelow, a detailed description will be given of embodiments of a medical information processing apparatus and a medical information processing system by referring to the accompanying drawings.

In one embodiment, a medical information processing apparatus includes processing circuitry. The processing circuitry acquires a plurality of medical support information items regarding a patient and a generation-time information item from a patient device to be operated by the patient, the generation-time information item indicating respective clock times at which the medical support information items are generated, generates timeline data indicating a temporal flow of generation of the medical support information items regarding the patient by arranging the medical support information items in accordance with the generation-time information item, and displays the timeline data on an online-medical-care support screen for supporting online medical care that is performed via the patient device and a doctor device to be operated by a doctor.

### First Embodiment

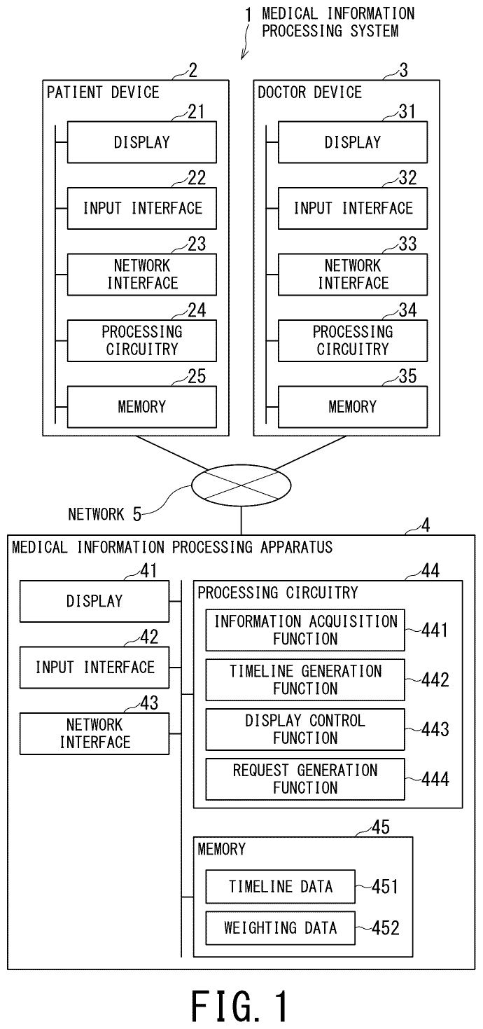

FIG. 1 is a block diagram illustrating a configuration of a medical information processing system 1 according to the first embodiment. The medical information processing system 1 is one example of an online-medical-care support system, and can be used not only for online medical care but also for online medical-examination consultation.

As shown in FIG. 1, the medical information processing system 1 includes a patient device 2, a doctor device 3, and a medical information processing apparatus 4. The patient device 2, the doctor device 3, and the medical information processing apparatus 4 are configured to be able to communicate with each other via a network 5. In the following, it is assumed that data are transmitted and received between respective devices and/or components via the network 5, and the phrase “via the network 5” is omitted in the description of transmission/reception of data.

The patient device 2 is a client terminal to be operated by a patient. For example, the patient device 2 is composed of a relatively small terminal device such as a smartphone and a tablet terminal. As shown in FIG. 1, the patient device 2 includes a display 21, an input interface 22, a network interface 23, processing circuitry 24, and a memory 25.

The display 21 of the patient device 2 is composed of a general display output device such as a liquid crystal display and an OLED (Organic Light Emitting Diode) display, for example. The input interface 22 is composed of a general input device such as a touch panel, and outputs an operation input signal corresponding to the patient's operation to the processing circuitry 24. The network interface 23 implements various information communication protocols depending on the type of the network 5. The network interface 23 connects the patient device 2 to the medical information processing apparatus 4 via the network 5 on the basis of the various protocols.

The processing circuitry 24 implements a function of integrally controlling the patient device 2. The processing circuitry 24 is a processor that reads out and executes programs stored in the memory 25 so as to perform processing in response to the patient's operation and processing in synchronization with the medical information processing apparatus 4, for example.

The memory 25 includes a recording medium that can be read by the processor, as exemplified by: a semiconductor memory element such as a flash memory and a RAM (Random Access Memory); a hard disk; and an optical disk. The memory 25 stores programs and parameter data to be used by the processing circuitry 24, for example.

The doctor device 3 is a client terminal to be operated by a doctor. The doctor device 3 is composed of a general information processing device such as a personal computer and a workstation. As shown in FIG. 1, the doctor device 3 includes a display 31, an input interface 32, a network interface 33, processing circuitry 34, and a memory 35.

The display 31 of the doctor device 3 is composed of a general display output device such as a liquid crystal display and an OLED display, for example. The input interface 32 is composed of general input devices such as a trackball, a switch button, a mouse, a keyboard, and a numeric keypad. The input interface 32 outputs an operation input signal corresponding to the doctor's operation to the processing circuitry 24. The network interface 33 implements various information communication protocols depending on the type of the network 5. The network interface 33 connects the doctor device 3 to the medical information processing apparatus 4 via the network 5 on the basis of the various protocols.

The processing circuitry 34 implements a function of integrally controlling the doctor device 3. The processing circuitry 34 is a processor that reads out and executes programs stored in the memory 35 so as to perform processing in response to the doctor's operation and processing in synchronization with the medical information processing apparatus 4, for example.

The memory 35 includes a recording medium that can be read by the processor, as exemplified by: a semiconductor memory element such as a flash memory and a RAM; a hard disk; and an optical disk. The memory 35 stores programs and parameter data to be used by the processing circuitry 34, for example.

The medical information processing apparatus 4 is a web server that generates web screens to be displayed on each of the patient device 2 and the doctor device 3. For example, the medical information processing apparatus 4 is composed of a general information processing apparatus such as a personal computer and a workstation. As shown in FIG. 1, the medical information processing apparatus 4 includes a display 41, an input interface 42, a network interface 43, processing circuitry 44, and a memory 45.

The display 41 of the medical information processing apparatus 4 is composed of a general display output device such as a liquid crystal display and an OLED display, for example. The input interface 42 is composed of general input devices such as a trackball, a switch button, a mouse, a keyboard, and a numeric keypad. The input interface 42 outputs an operation input signal corresponding to an operation by a user such as a web-server operator to the processing circuitry 44. The network interface 43 implements various information communication protocols depending on the type of the network 5. The network interface 43 connects the medical information processing apparatus 4 to the patient device 2 and the doctor device 3 via the network 5 on the basis of the various protocols.

The processing circuitry 44 implements a function of integrally controlling the medical information processing apparatus 4. The processing circuitry 44 is a processor that reads out and executes programs stored in the memory 45 so as to execute processing of generating and updating the web screens.

The processor of the processing circuitry 24 implements an information acquisition function 441, a timeline generation function 442, a display control function 443, and a request generation function 444. These functions are stored in the memory 45 in the form of programs, for example.

The information acquisition function 441 includes a function to acquire a plurality of patient information items on the patient and a generation-time information item indicating the clock time (i.e., time point) of completing generation of each of the patient information items from the patient device 2 to be operated by the patient. The patient information items are information that helps the doctor treat the patient. For example, the patient information items include: vital signs (consciousness, respiration, body temperature, degree of blood oxygen saturation, pulse, and blood pressure), a symptom, a facial expression, a body condition, and mood of the patient. Each patient information item is one example of a medical support information item.

The timeline generation function 442 includes a function to generate timeline data indicating the temporal flow of generation of the patient information items by arranging the patient information items in accordance with the generation-time information item.

The display control function 443 includes a function to display the timeline data on an online-medical-care support screen for supporting the online medical care, which is performed through the patient device 2 to be operated by the patient and the doctor device 3 to be operated by the doctor.

The request generation function 444 may include at least one of: a function to generate a request indicating a patient information item that the doctor wants to acquire from the patient as information necessary for medical care; and a function to analyze a trend in symptoms of the patient on the basis of the timeline data.

The memory 45 includes a recording medium that can be read by the processor, as exemplified by: a semiconductor memory element such as a flash memory and a RAM; a hard disk; and an optical disk. The memory 45 stores programs and parameter data to be used by the processing circuitry 44, for example.

The memory 45 stores at least timeline data 451 and weighting data 452. The timeline data 451 include a time axis, one or plural patient information items associated with a predetermined date and time on the time axis, and an icon indicating the existence of the patient information item, for example. The weighting data 452 are data that indicate cautions or notes regarding weighting of each request and the patient information items. The weighting data 452 are set on the basis of the evaluation of the request and the patient information item(s), as exemplified by evaluation of validity and contribution to diagnosis of the patient. For example, the weighting data 452 are a priority order of requests generated in a predetermined situation, display (e.g., an icon) for calling attention to a specific patient information item and so on.

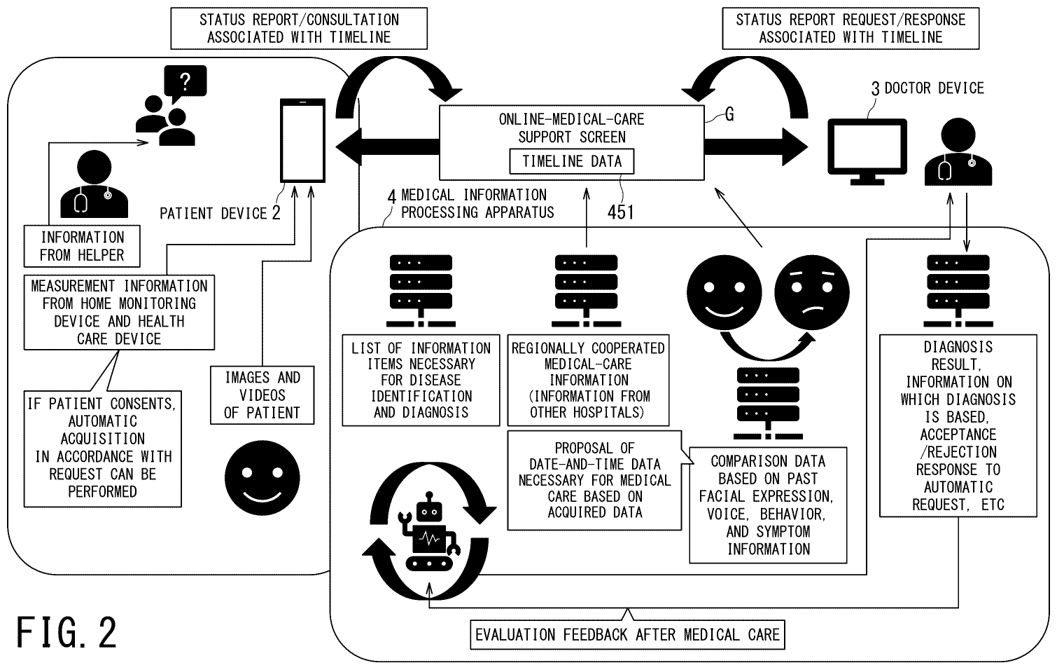

FIG. 2 is a schematic diagram illustrating an outline of processing to be performed by the medical information processing system 1 according to the first embodiment. As shown in FIG. 2, the patient device 2 and the doctor device 3 share the online-medical-care support screen G including the timeline data 451, through the medical information processing apparatus 4. In other words, in the patient device 2, the online-medical-care support screen G is displayed on the display 21, and first additional information (for example, a status report) can be added or uploaded to the timeline data 451 by the patient's operation on the input interface 22. In the doctor device 3, the online-medical-care support screen G is displayed on the display 31, and second additional information (for example, a request) can be added to the timeline data 451 by the doctor's operation on the input interface 32.

The additional information added to the timeline data 451 from one of the patient device 2 and the doctor device 3 is reflected in real time in the timeline data 451 shown in the other device via the medical information processing apparatus 4.

For example, the doctor adds (i.e., sends) a request for a status report to the patient in the timeline data 451 displayed on the doctor device 3 regarding the status at a specific date and time that are considered to be important for diagnosis. The patient refers to the request from the doctor in the timeline data 451 displayed on the patient device 2, and adds (i.e., submits) the status report in the timeline data 451 in response to the doctor. The doctor refers to the status report from the patient in the timeline data 451 displayed on the doctor device 3. The status report is one example of the medical support information item and the patient information item.

The status report may include not only the patient's chief complaint in text but also images, videos, voice data, and photograph information that are stored in a device such as the patient's smartphone or acquired in real time. This configuration brings the online medical care closer to face-to-face medical care, and enables comparison between the patient at present and the patient at ordinary times. In addition, the status report may include measurement information items acquired from an activity-condition monitoring device and/or an arbitrary biosensor device (health care device) as well as information acquired from a helper such as a cohabitant.

Under the condition that the patient's consent is acquired, when the patient device 2 receives a request for the status report from the doctor, the patient device 2 may collect measurement information items from respective devices in accordance with the request and add the collected measurement information items to the timeline data 451 without any operation by the patient.

In the timeline data 451 displayed on the patient device 2, the patient adds a request for consultation, a question, supplementary explanation, and so on to the doctor to the date and time corresponding to that present time. The doctor refers to the patient's request in the timeline data 451 displayed on the doctor device 3, and adds a response addressed to the patient to the timeline data 451. The patient refers to the doctor's response in the timeline data 451 displayed on the patient device 2. This configuration enables mutual understanding between the patient and the doctor comparable to a face-to-face level.

The medical information processing apparatus 4 stores a list of information items necessary for disease identification and diagnosis, regionally cooperated medical care information (for example, information on the patient diagnosed in another hospital), and comparison data of facial expressions, voices, behaviors, and symptoms in the past, for example. The medical information processing apparatus 4 then acquires the patient information items in chronological order and analyzes a trend in patient symptoms by using the stored data and a model of AI (Artificial Intelligence, e.g., a machine learning model such as a neural network), for example. Further, the medical information processing apparatus 4 generates a request for an additional information item (i.e., data on a specific date and time) that is necessary for identifying a high-risk disease, and proposes this request to the doctor. This configuration enables quality of a medical consultation and examination equivalent to or higher than that of a face-to-face consultation.

Note that both the patient device 2 and the doctor device 3 may limit the scope of disclosure of the information added to the timeline data 451 as appropriate by specifying a mask depending on the other party.

After the medical care is completed, the medical information processing apparatus 4 further acquires an evaluation result of the request of the status report from the doctor device 3 and then feeds back the evaluation result to the memory 45. The evaluation result includes evaluation of: a diagnosis result, information on which the diagnosis is based, and an acceptance/rejection response to the request generated by the medical information processing apparatus 4. This configuration allows the medical information processing apparatus 4 to generate requests in line with the doctor's thinking.

Even if the generated request itself is valid, there are cases where the patient's status report in response to this generated request is invalid or NG. In such a case, the medical information processing apparatus 4 further acquires information on the validity of the status report to the request from the doctor device 3 and feeds this information back to the model of AI. This configuration allows the medical information processing apparatus 4 to generate requests in line with the doctor's thinking. Note that the above-described validity is one aspect of the evaluation.

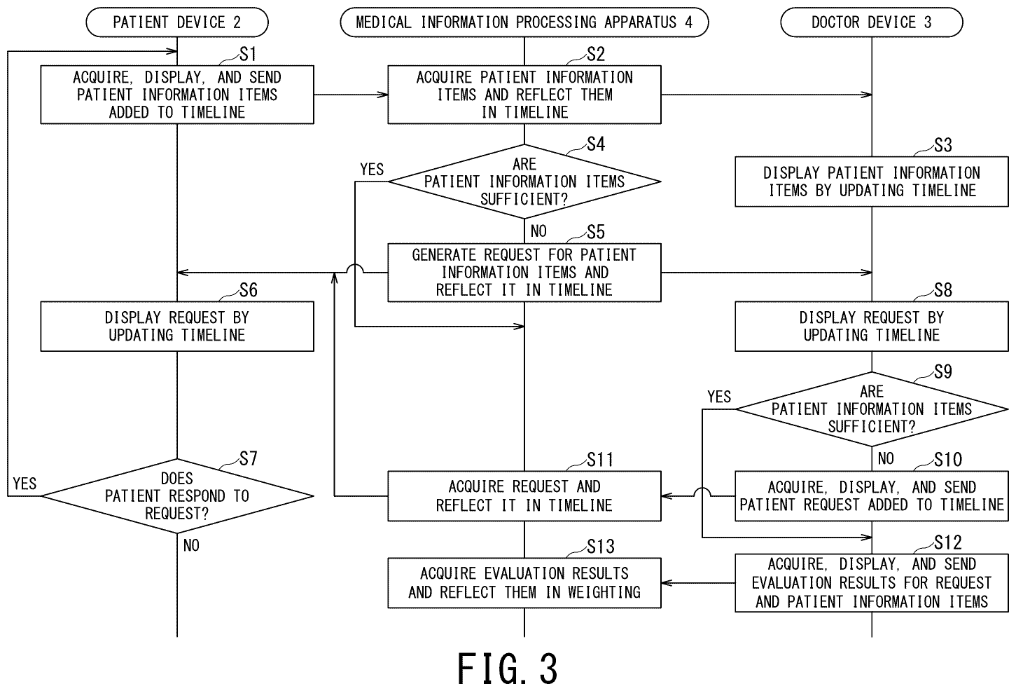

FIG. 3 is a sequence chart illustrating processing to be performed by the medical information processing system 1 according to the first embodiment. This processing embodiment describes inputs to the timeline data 451 and their reflection. In particular, a description will be given of the processing of the patient device 2, the medical information processing apparatus 4, and the doctor device 3 as well as data exchange between these three.

In the step S1, the patient adds the patient information items to the timeline data 451 on the online-medical-care support screen G displayed on the display 21 by operating the input interface 22 of the patient device 2. The patient adds a symptom that the patient has noticed, such as change in his/her physical condition, to the timeline data 451. The patient can select physical activity data acquired from an arbitrary healthcare device installed in his/her home and attach the physical activity data to the report addressed to the doctor. The physical activity data include the number of steps taken, sleeping hours, condition of turning over in bed, tone of voice, a facial expression, complexion, frequency of using the toilet, for example.

At this time, the processing circuitry 24 of the patient device 2 acquires the patient information items from the input interface 22 and displays the patient information items on the timeline data 451 of the display 21. The processing circuitry 24 then transmits the patient information items to the medical information processing apparatus 4.

The patient device 2 may acquire not only the patient information items but also information on the patient's residential environment at the time of the medical examination (such as temperature, humidity, atmospheric pressure, dust concentration, presence/absence of a pet, and type of a pet), display the residential environment information on the timeline data 451, and transmit the residential environment information to medical information processing apparatus 4.

In the step S2, the information acquisition function 441 of the medical information processing apparatus 4 acquires the patient information items from the patient device 2 via the network interface 43, and reflects the patient information items in the timeline data 451 of the memory 45. At this time point, the state in which these patient information items are reflected is the latest state of the timeline data 451.

In the step S3, the processing circuitry 34 of the doctor device 3 updates the timeline data 451 displayed on the display 31 so as to display the patient information items on the timeline data 451. “Updates the timeline data 451” means to bring the timeline data 451 of the display 31 to the latest state of the timeline data 451 stored in the memory 45 of the medical information processing apparatus 4.

In the step S4, the processing circuitry 44 of the medical information processing apparatus 4 determines whether the patient information items in the timeline data 451 are sufficient or not. For example, when a description of a predetermined symptom is included in the patient information items, the processing circuitry 44 determines whether the patient information items include sufficient information necessary for diagnosing the symptom or not. If the patient information items are sufficient (YES in the step S4), the processing circuitry 44 skips the step S5 and waits. If the patient information items are not sufficient (NO in step S4), the processing circuitry 44 advances the processing to the step S5.

In the step S5, the request generation function 444 of the medical information processing apparatus 4 generates a request for a patient information item on the basis of the weighting data 452, and reflects this request in the timeline data 451. At this time point, the state in which this request is reflected is the latest state of the timeline data 451.

On the basis of information items organized in chronological order, such as a trend in patient symptoms, the request generation function 444 generates a request for the patient information item that needs to be added in order to identify a high-risk disease (or to recommend a face-to-face examination, for example). At this time, the request generation function 444 determines the time point or clock time at which the changes in symptoms and/or other matters should be checked according to the trend of the patient, and specifies a specific date and time in the past in the generated request. Further, at the position indicating the specified date and time in the timeline data 451 of the online-medical-care support screen G, the display control function 443 displays the request for the patient information item that needs to be added.

In the step S6, the processing circuitry 24 of the patient device 2 updates the timeline data 451 displayed on the display 21 so as to display the request on the timeline data 451. “Updates the timeline data 451” means to bring the timeline data 451 of the display 21 to the latest state of the timeline data 451 stored in the memory 45 of the medical information processing apparatus 4.

In the step S7, the processing circuitry 24 of the patient device 2 determines whether the patient responds to the request or not. For example, the processing circuitry 24 determines whether the input interface 22 has been operated within a predetermined period of time after displaying the request or not. If the patient responds to the request (YES in the step S7), the processing circuitry 24 returns the processing to the step S1. If the patient does not respond to the request (NO in the step S7), the processing circuitry 24 waits.

The steps S6 and S7 are executed not only in response to the request from the medical information processing apparatus 4 but also in response to the request from the doctor device 3 (i.e., the processing from the steps S10 and S11 to the steps S6 and S7).

In the step S8, the processing circuitry 34 of the doctor device 3 updates the timeline data 451 displayed on the display 31 so as to display the request in the timeline data 451.

In the step S9, the processing circuitry 34 of the doctor device 3 determines whether the patient information items in the timeline data 451 shown on the display 31 are sufficient or not. For example, the processing circuitry 34 determines whether the input interface 32 has been operated within a predetermined period of time after displaying the patient information items or not. If this operation is not performed within the predetermined period of time, it can be said that the patient information items are sufficient for the doctor. If this operation is performed within the predetermined period of time, it can be said that the patient information items are not sufficient for the doctor. If the patient information items are sufficient (YES in the step S9), the processing circuitry 34 skips the step S10 and waits. If the patient information items are not sufficient (NO in the step S9), the processing circuitry 34 advances the processing to the step S10.

In the step S10, the doctor adds a request for the patient information item to the timeline data 451 displayed on the display 31 of the doctor device 3 by operating the input interface 32. For example, the doctor may request supplementary explanation from the patient regarding specific acquired data (specific difference between the current data and data at a specific time point). This allows detailed information to be collected in chronological order.

At this time point, the processing circuitry 34 of the doctor device 3 acquires the doctor's request from the input interface 32, and displays the request on the timeline data 451 of the display 31. The network interface 33 then transmits the request to the medical information processing apparatus 4.

In the step S11, the information acquisition function 441 of the medical information processing apparatus 4 acquires the request from the doctor device 3 via the network interface 43, and reflects this request in the timeline data 451 in the memory 45. At this time point, the state in which this request is reflected is the latest state of the timeline data 451.

Afterward, the processing of the steps S6 and S7 is performed.

In the step S12, after the medical care for the patient is completed, the doctor inputs an evaluation result of the request generated by the medical information processing apparatus 4 or an evaluation result of the patient information item in response to the request to the online-medical-care support screen G by operating the input interface 32. For example, in response to the proposal for the additional information request from the medical information processing apparatus 4, the doctor evaluates the validity of the request after completing the diagnosis, and inputs the evaluation result into the input interface 32 of the doctor device 3.

At this time, the processing circuitry 34 of the doctor device 3 acquires the evaluation result by the doctor from the input interface 32, and then displays the evaluation result on the online-medical-care support screen G of the display 31. The processing circuitry 34 then transmits the evaluation results to the medical information processing apparatus 4.

In the step S13, the information acquisition function 441 of the medical information processing apparatus 4 acquires the evaluation result of the request and the evaluation result of the patient information item from the doctor device 3 via the network interface 43, and reflects these evaluation results in the weighting data 452 stored in the memory 45. The evaluation result of the request is one example of the first evaluation information indicating the evaluation of the request. The evaluation result of the patient information item is one example of the second evaluation information that indicates the evaluation of the patient information item added in response to the request.

The request generation function 444 updates the weighting of the evaluated request on the basis of the evaluation result of this request. The request generation function 444 may adjust the priority order of the patient information items when generating a request in the subsequent medical care, for example.

The request generation function 444 updates the weighting of the added patient information item on the basis of the evaluation result of the added patient information item. For example, if the patient's response is not in accordance with the doctor's intention, the request generation function 444 may store data indicating invalidity of the response in the memory 45 so as to improve the expression of future requests on the basis of these data. If the authenticity of the patient's response (i.e., the patient information item provided in response to the request) is questionable, the display control function 443 may add a warning sign (such as an icon and a pop-up showing a supplementary comment from the doctor) to this patient information item on the timeline data 451.

The medical information processing system 1 may be configured such that the doctor can select whether or not to notify the patient of the request displayed in the step S8. In this case, after generating the request, the medical information processing apparatus 4 transmits data indicating the contents of the request to the doctor device 3 without reflecting the request in the timeline data 451, and the doctor device 3 displays the contents of the request on the display 31 after receiving these data from the medical information processing apparatus 4. The doctor device 3 acquires a response indicating whether the doctor accepts the request or not via the input interface 32, and sends a message regarding the response to the medical information processing apparatus 4. The medical information processing apparatus 4 receives the message from the doctor device 3. If this message indicates that the doctor accepts the request, the medical information processing apparatus 4 reflects the request in the timeline data 451 in the memory 45.

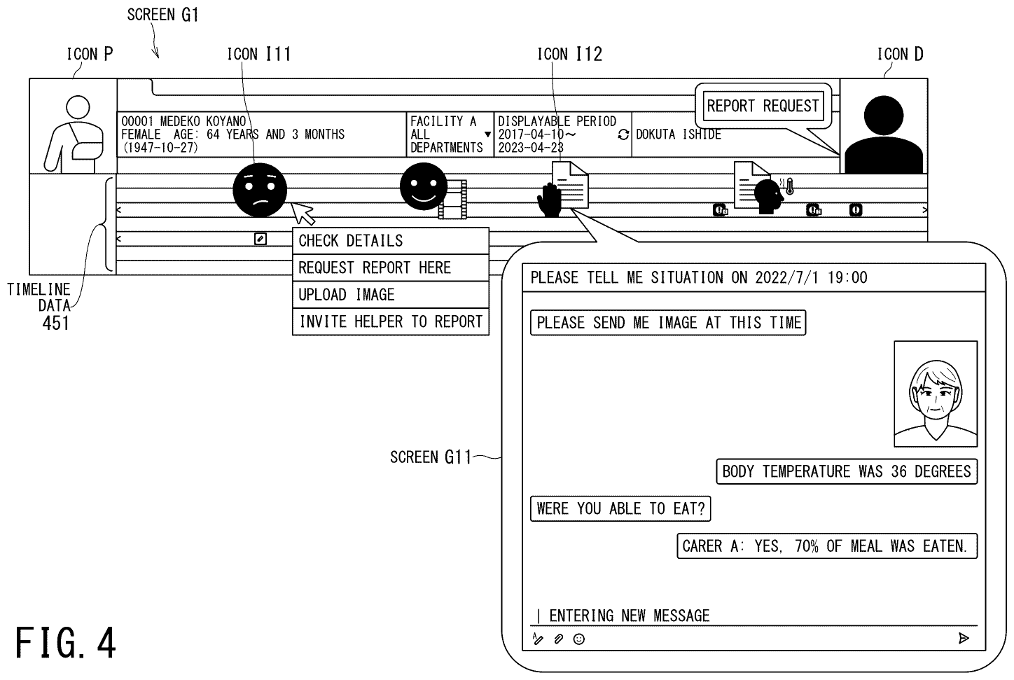

FIG. 4 illustrates the online-medical-care support screen according to the first embodiment. The screen G1 is one example of the online-medical-care support screen to be displayed on a tablet and/or a personal computer. On the screen to be displayed on a smartphone, timeline data (time-series portions) are displayed, and if the icon is tapped, a pop-up window showing the details appears over the entire screen. In principle, the same screen G1 is displayed on the display 21 of the patient device 2 and the display 31 of the doctor device 3. However, in some cases, a portion that should be seen only by the doctor is subtly masked at the patient's end. Conversely, in some cases, a portion that should be seen only by the patient is subtly masked at the doctor's end. In addition, the screen G1 includes sections requiring the patient to fill in and sections requiring the doctor to fill in.

As shown in FIG. 4, an icon P on the left represents the patient. An icon D on the right represents the doctor. Between the icon P and the icon D, information items such as a patient's name, a facility being used, a name of the diagnosis and treatment department, a displayable period, and a doctor's name are displayed from the left side. Additionally, a plurality of icons are added to the timeline data 451 below the aforesaid information items. Each icon indicates that a patient information item has been added to the date and time corresponding to the position of the icon on the timeline data 451.

For example, when the patient or the doctor selects an icon I11 and then right-clicks, the patient or the doctor can select any one of “check details”, “request report here”, “upload image”, and “invite helper to report”.

When “check details” is selected, details of the patient information items associated with the icon I11 are displayed.

When “request report here” is selected, a new item can be entered for requesting a report related to the patient information items associated with the icon I11.

When “upload image” is selected, image data related to the patient information items associated with the icon I11 can be uploaded.

When “invite helper to report” is selected, the user can enter the identification information of the helper that the user would like to cooperate with in reporting the patient information items associated with icon I11.

When the doctor selects an icon I12 and then right-clicks “request report here”, the screen G11 pops up. The top of the screen G11 displays a message of “Please tell me the situation on” and “date and time” at which the icon I12 is added. Afterwards, each time a new message is inputted, the doctor's message is displayed on the left side of the screen G11 or the patient's message is displayed on the right side of the screen G11.

When the patient or the doctor selects the icon I12 and then right-clicks “invite helper to report”, the screens G1 and G11 are also shared with the helper C. Further, a message inputted by the helper C is displayed on the screen G11.

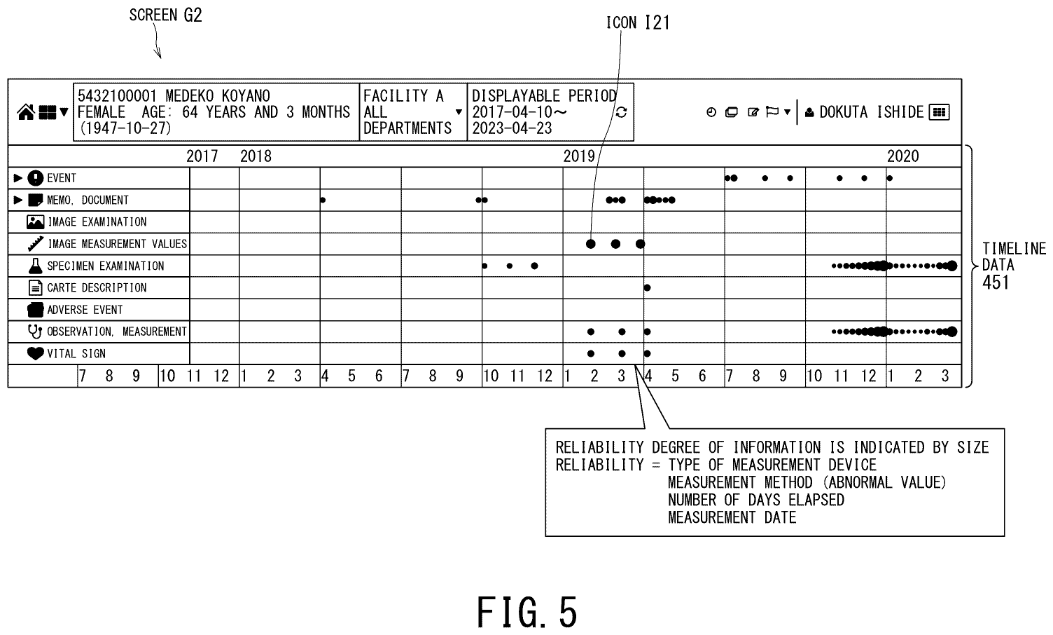

FIG. 5 illustrates a case of showing a reliability degree of each patient information item on the online-medical-care support screen according to the first embodiment. The screen G2 is one example of the online-medical-care support screen.

For example, an icon I21 is displayed as a black circle. The size of the icon I21 (for example, the diameter of circle) to be displayed in the timeline data 451 is specified corresponding to the reliability degree of the patient information item associated with the icon I21. The higher the reliability degree of the patient information item is, the larger the size of the icon I21 becomes. The color of the icon I21 may be specified corresponding to the reliability degree of the patient information item.

The reliability degree of the patient information item is adjusted depending on a type of measurement device, a measurement method, number of days elapsed, and measurement date, for example. Regarding the type of measurement device that is the source of information, high reliability degree values are set for official medical devices, whereas low reliability degree values are set for non-medical devices such as a smart watch. In the medical information processing apparatus 4, the memory 45 stores reliability degree values for the respective types of measurement devices. The memory 45 may store the reliability degree values in a database storage format (for example, a relational database).

Regarding the measurement method, high reliability degree values are set for measurements performed by a nurse or a carer, whereas low reliability degree values are set for simple measurement methods that anyone can perform.

Regarding the number of days elapsed, the more time elapses from the acquisition of the patient information item, the lower reliability degree of this patient information item becomes. The time point (i.e., clock time) at which the patient information item is uploaded to the timeline data 451 is treated as the time point (i.e., clock time) at which the patient information item is acquired.

Regarding the measurement date, the surrounding environment and location of the patient on that measurement day are considered important. When the patient is resting at home on that measurement day, a high reliability degree value is set. Conversely, for example, when the patient is staying outdoors on that measurement day, a low reliability degree value is set.

Each icon indicating the patient information item acquired in response to the request generated by the medical information processing apparatus 4 is necessary for identifying the disease of the patient, and thus, such icons are displayed in a particularly conspicuous manner.

Furthermore, outliers (i.e., invalid values) are defined based on the assumption that the measured value of a sensor deviates due to a body motion of the patient. The memory 45 stores the defined outliers. The processing circuitry 44 performs a comparison determination between each of the measured data and the corresponding outlier in the memory 45. If the actual measured value outputted by the sensor matches the outlier or is within the range of the outlier, the processing circuitry 44 does not add this measured value to the timeline data 451. In addition, the processing circuitry 44 may display this measured value in a color that enables to identify this measured value as an abnormal or invalid value on the screen G2.

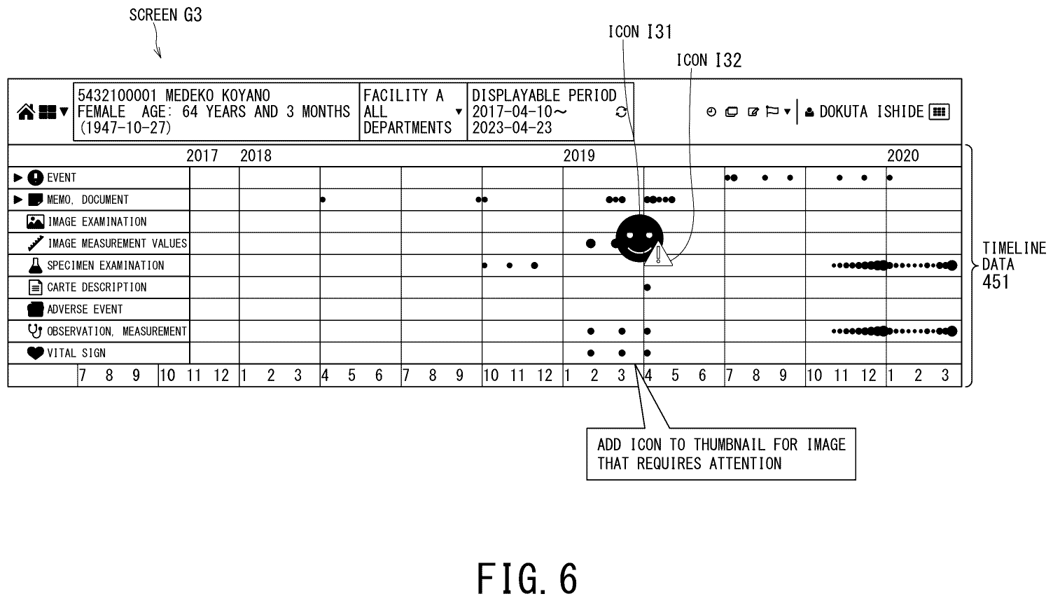

FIG. 6 illustrates another case of showing the reliability degree of each patient information item on the online-medical-care support screen according to the first embodiment. The screen G3 is one example of the online-medical-care support screen. For example, when the patient information item associated with the icon I31 includes an image or video that requires attention, the display control function 443 of the medical information processing apparatus 4 additionally displays an icon I32 for calling attention to the icon I31. Instead of the icon I31, a thumbnail indicative of an overview of the patient information item may be displayed.

For example, images that require attention include: an image that may be out of focus based on AI analysis; and an image that may be in a difficult condition to evaluate, such as an image with insufficient light, a backlight image, or an image that has not been subjected to camera-shake compensation, based on a determination result of the imaging condition derived from an EXIF (Exchangeable Image File Format) tag and so on.

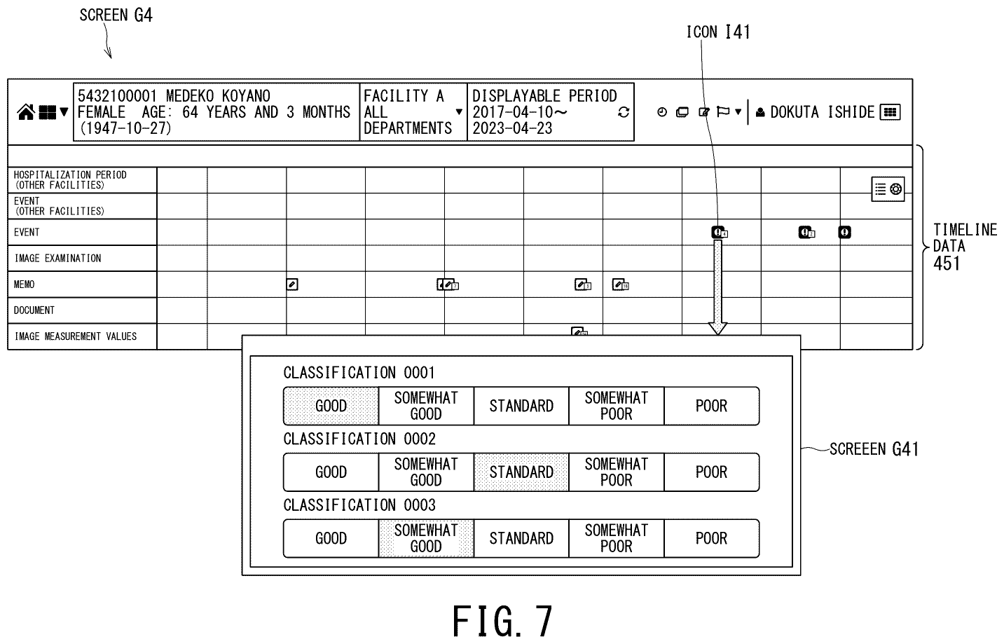

FIG. 7 illustrates a case of showing a contribution degree of each patient information item on the online-medical-care support screen according to the first embodiment. The contribution degree is one example of evaluation. A screen G41 is one example of the online-medical-care support screen. When a predetermined operation is performed with respect to the icon I41, the screen G41 pops up. The predetermined operation is, for example, to select the icon I41 and then left-click “classification evaluation” from the options displayed by right-clicking the mouse.

With respect to the patient diagnosis, the screen G41 allows input or selection of the contribution degree of the patient information item (especially, measurement information, patient images, and videos) associated with the icon I41. Classification 000X may be assigned to each doctor, may be assigned to each diagnosis and treatment department for a large-scale hospital, or may be assigned to each doctor-patient pair for home medical care. In other words, the evaluation result of the patient information item indicates the validity of this patient information item for each diagnosis and treatment department, for each doctor, or for each doctor-patient pair.

The processing circuitry 44 of the medical information processing apparatus 4 registers the contribution degrees of respective patient information items for each doctor in the memory 45. Further, the processing circuitry 44 causes the memory 45 to update the weighting data 452 according to the contribution degrees of the respective patient information items. In other words, on the basis of the newly acquired evaluation result of the patient information item, the request generation function 444 updates the weighting of the patient information items for each diagnosis and treatment department, for each doctor, or for each doctor-patient pair.

For example, when a doctor A selects “the contribution degree of the measurement information by the sensor B is good” or “somewhat good”, the processing circuitry 44 increases the weighting of the measurement information by the sensor B for the doctor A in the weighting data 452.

Conversely, when the doctor A selects “the contribution degree of the measurement information by the sensor C is somewhat poor” or “poor”, the processing circuitry 44 reduces the weighting of the measurement information by the sensor C for the doctor A in the weighting data 452.

When the doctor A selects “the contribution degree of the measurement information by the sensor D is standard”, the processing circuitry 44 does not update the weighting of the measurement information by the sensor D for the doctor A in the weighting data 452.

By the above-described weighting update, the request generation function 444 of the medical information processing apparatus 4 can generate requests according to the doctor's individual preferences by referring to the weighting data 452 of the memory 45. The above-described weighting update allows variations in all the units to which doctor's evaluations should be fed back, such as each doctor, each diagnosis and treatment department in a large hospital, and each doctor-patient pair.

### Effects of First Embodiment

Even when a patient visits a medical institution that has little medical-examination history of this patient and lacks sufficient face-to-face medical treatment data of this patient in the past, this patient and the doctor can share the past medical information and the body condition when being healthy in the past in chronological order. Since the patient and the doctor are looking at the same screen, it becomes easier for the patient to clearly specify a question, and the doctor can understand the patient's question better. This configuration enables communication between the patient and the doctor in a manner similar to face-to-face medical care at the family doctor's clinic. This configuration can expand the range of diagnosis of illnesses and injuries through the online medical care, and can reduce distance and time constraints.

In addition, even if the patient cannot communicate his/her symptoms verbally, the doctor can intuitively understand the patient's situation by using the image data such as still image data and video data on the timeline data 451. Further, even in the case of intermittent symptoms that cannot be observed at the time of visiting the clinic, when the patient adds data such as image data and measurement data to the corresponding date and time on the timeline data 451 at home, the doctor can check the symptoms. Moreover, adding a patient information item and a request at a predetermined time point (i.e., clock time) on the timeline data 451 makes it easier to know the chronological history of interaction between the patient and the doctor, and contributes to improvement in communication.

### Second Embodiment

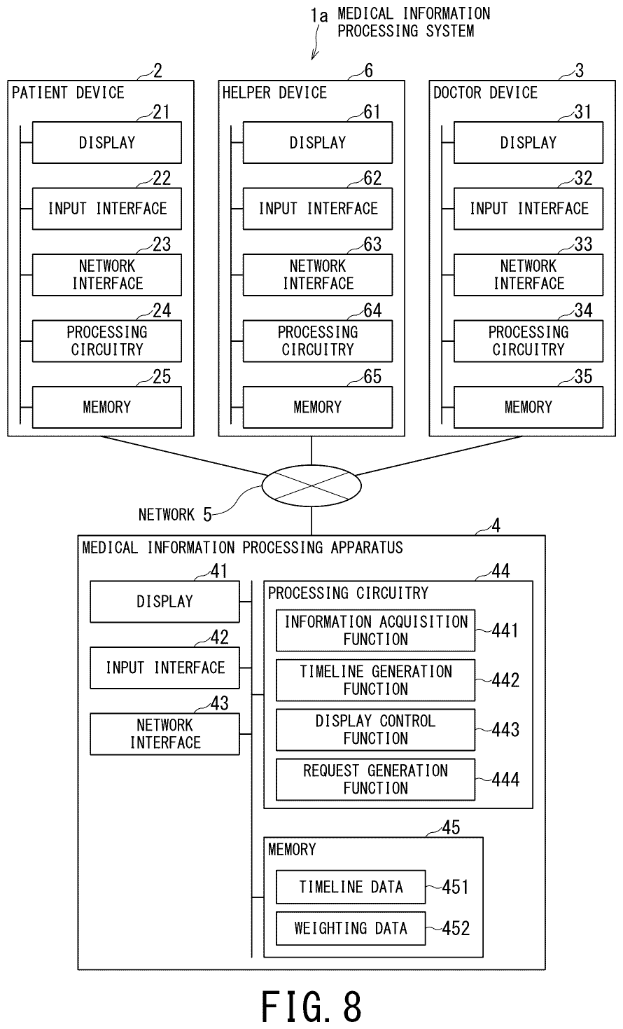

FIG. 8 is a block diagram illustrating a configuration of a medical information processing system 1a according to the second embodiment. The medical information processing system 1a is one example of the online medical care support system, and can be used not only for the online medical care but also for online medical-examination consultation. As shown in FIG. 8, the medical information processing system 1a includes the patient device 2, the doctor device 3, the medical information processing apparatus 4, and a helper device 6. The patient device 2, the doctor device 3, the medical information processing apparatus 4, and the helper device 6 are configured to be able to communicate with each other via the network 5.

The helper device 6 is a client terminal to be operated by a helper. The helper is a person who assists a patient, such as a person living with the patient at the patient's home and a carer (i.e., caregiver) who provides care services. The helper device 6 is composed of a relatively small terminal device such as a smartphone and a tablet terminal. As shown in FIG. 8, the helper device 6 includes a display 61, an input interface 62, a network interface 63, processing circuitry 64, and a memory 65.

The display 61 of the helper device 6 is composed of a general display output device such as a liquid crystal display and an OLED display, for example. The input interface 62 is composed of a general input device such as a touch panel, and outputs an operation input signal corresponding to the helper's operation to the processing circuitry 64. The network interface 63 implements various information communication protocols depending on the type of the network 5. The network interface 63 connects the helper device 6 to the medical information processing apparatus 4 via the network 5 on the basis of the various protocols.

The processing circuitry 64 implements a function of integrally controlling the helper device 6. The processing circuitry 64 is a processor that performs processing of receiving the helper's operation by reading out and executing programs stored in the memory 65.

The memory 65 includes a recording medium that can be read by the processor, as exemplified by: a semiconductor memory element such as a flash memory and a RAM; a hard disk; and an optical disk. The memory 65 stores programs and parameter data to be used by the processing circuitry 64, for example.

In the medical information processing apparatus 4, the information acquisition function 441 acquires both the medical support information items related to the patient or the helper and the generation-time information item indicating the respective click times (i.e., time points) of completing generation of the medical support information items, from the helper device 6 to be operated by the helper. The helper is one example of a third person.

The timeline generation function 442 generates the timeline data 451 indicating the temporal flow of generation of the medical support information items regarding the patient or the helper by arranging the medical support information items in accordance with the generation-time information item, and stores the timeline data 451 in the memory 45. Further, the display control function 443 displays the timeline data 451 on the online-medical-care support screen G meant for supporting the online medical care provided through the patient device 2, the doctor device 3, and the helper device 6.

Since the helper device 6 can be operated by the helper, the helper can communicate with the doctor together with the patient. For example, the doctor can acquire information on the patient from a cohabitant in a separate window through hearing. In other words, the doctor can acquire information from an outsider's perspective. This configuration allows the doctor to collect a wide range of information on the patient and thus contributes to patient care.

### Third Embodiment

The third embodiment relates to a configuration in which the medical information processing apparatus 4 detects an abnormal condition of the patient and notifies the doctor of the abnormal condition.

### (1) Acceleration Sensor

Measured value data may be acquired from an acceleration sensor attached to the patient. In this case, the information acquisition function 441 of the medical information processing apparatus 4 acquires the measured value data from the acceleration sensor through the patient device 2 and stores the measured value data in the memory 45. The request generation function 444 then analyzes the measured value data in the memory 45 over a predetermined period, and thereby predicts an abnormality, such as decrease in the patient's walking speed and gait abnormality, on the basis of change in acceleration, for example. The display control function 443 reflects the abnormal condition in the timeline data 451.

### (2) Microphone

The measured value data may be acquired from a microphone such as a headset worn by the patient. In this case, the information acquisition function 441 of the medical information processing apparatus 4 acquires the patient's voice data collected by the microphone via the patient device 2, and stores the voice data in the memory 45. The request generation function 444 then analyzes the voice data in the memory 45 over a predetermined period of time, and thereby predicts an abnormal condition of the patient on the basis of change in voice of the patient. The request generation function 444 predicts an abnormal condition of the patient by detecting: change in the content of the patient's utterances (such as frequently used words and frequent topics in the conversation); and change in the fluency of speech (e.g., the patient may gradually have difficulty saying a understandable sentence), for example. The display control function 443 reflects the abnormal condition in the timeline data 451.

### (3) Camera

Image data may be acquired from a camera placed at a position where the camera can image the patient's dining table. In this case, the information acquisition function 441 of the medical information processing apparatus 4 acquires image data indicative of the patient's state during a meal imaged by the camera through the patient device 2, and stores the image data in the memory 45. The request generation function 444 then analyzes the image data in the memory 45 over a predetermined period of time, and thereby predicts an abnormal condition of the patient on the basis of changes in diet. The request generation function 444 predicts the abnormal condition of the patient by detecting change in the content of the patient's meals, the amount of food consumed by the patient, and eating speed of the patient, for example. The display control function 443 reflects the abnormal condition in the timeline data 451.

Note that the image data may be acquired from a camera installed at a position where this camera can image the patient's daily life. For example, by this configuration, if the order of actions in the daily life changes, the request generation function 444 predicts that the dementia is worsening or that the patient cannot perform usual activities due to fatigue. Additionally, the request generation function 444 may analyze change in sleeping hours and condition of turning over in bed by using a sensor attached to the patient.

Further, the medical information processing apparatus 4 may store data such as still images, videos, and voices of the patient in the memory 45 via the patient device 2 so as to extract temporal difference in the data without predicting the abnormal condition of the patient. The medical information processing apparatus 4 may then propose and display temporal change in the condition of the patient on the online-medical-care support screen G in chronological order.

The medical information processing apparatus 4 may receive a request indicating a specific patient information item, which the doctor wants to know, from the doctor device 3 so as to acquire the data corresponding to this request from the patient device 2. The medical information processing apparatus 4 may reflect the acquired data and its analysis result in the timeline data 451.

According to at least one embodiment described above, in the online medical care, shortage of patient information that the doctor needs can be alleviated.

While certain embodiments have been described, these embodiments have been presented by way of example only, and are not intended to limit the scope of the invention. Indeed, the novel methods and systems described herein may be embodied in a variety of other forms; furthermore, various omissions, substitutions, changes, and combinations of embodiments in the form of the methods and systems described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to:
acquire a plurality of medical support information items regarding a patient and a generation-time information item from a patient device to be operated by the patient, the generation-time information item indicating respective clock times at which the medical support information items are generated;
generate timeline data indicating a temporal flow of generation of the medical support information items regarding the patient by arranging the medical support information items in accordance with the generation-time information item; and
display the timeline data on an online-medical-care support screen for supporting online medical care that is performed via the patient device and a doctor device to be operated by a doctor.

2. The medical information processing apparatus according to claim 1, wherein the processing circuitry is configured to:
generate a request for a medical support information item; and
display the request for a medical support information item at a position indicating a predetermined clock time in the timeline data.

3. The medical information processing apparatus according to claim 2, wherein the processing circuitry is configured to:
analyze a trend of symptoms of the patient based on the timeline data;
generate a request for the medical support information item that needs to be added in order to identify a high-risk disease based on the trend of symptoms of the patient; and
display, on the online-medical-care support display, a request for the medical support information item that needs to be added.

4. The medical information processing apparatus according to claim 2, wherein the processing circuitry is configured to:
acquire first evaluation information indicating evaluation of the request from the doctor device; and
update weighting of the request based on the first evaluation information.

5. The medical information processing apparatus according to claim 2, wherein the processing circuitry is configured to:
acquire second evaluation information indicating evaluation of the medical support information item added in response to the request from the doctor device; and
update weighting of an added medical support information item based on the second evaluation information.

6. The medical information processing apparatus according to claim 5, wherein:
the second evaluation information indicates evaluation of the patient information item for each diagnosis and treatment department, for each doctor, or for each doctor-patient pair; and
the processing circuitry is configured to update weighting of the patient information item for each diagnosis and treatment department, for each doctor, or for each doctor-patient pair based on the second evaluation information.

7. The medical information processing apparatus according to claim 1, wherein the processing circuitry is configured to acquire both a medical support information item regarding the patient or a third person and a generation-time information item indicating a clock time of completing generation of the medical support information item from a helper device to be operated by the third person.

8. A medical information processing system comprising:
a patient device to be operated by a patient;
a doctor device to be operated by a doctor;
a medical information processing apparatus capable of communicating with the patient device and the doctor device,
wherein the medical information processing apparatus comprises processing circuitry configured to:
acquire, from the patient device, a plurality of medical support information items regarding the patient and a generation-time information item indicating respective clock times at which the medical support information items are generated;
generate timeline data indicating a temporal flow of generation of the medical support information items regarding the patient by arranging the medical support information items in accordance with the generation-time information item; and
display the timeline data on an online-medical-care support screen for supporting online medical care that is performed via the patient device and the doctor device.
